(19) United States
(12) Patent Application Publication
(10) Pub. No.: US 2007/0071819 A1
(43) Pub. Date: Mar. 29, 2007

(54) MULTIPLE UNIT MODIFIED RELEASE COMPOSITIONS OF CARBAMAZEPINE AND PROCESS FOR THEIR PREPARATION

(76) Inventors: Amit Kumar Kesarwani, Haridwar (IN); Manish Chawla, Rohini (IN); Rajeev Singh Raghuvanshi, Gurgaon (IN); Ashok Rampal, Amritsar (IN)

Correspondence Address: RANBAXY INC., 600 COLLEGE ROAD EAST, SUITE 2100, PRINCETON, NJ, 08540, US

(21) Appl. No.: 11/420,957

(22) Filed: May 30, 2006

(30) Foreign Application Priority Data
May 30, 2005 (IN) 1380/DEL/2005
May 30, 2005 (IN) 1382/DEL/2005

(51) Int. Cl. A61K 31/55; A61K 9/22; A61K 9/26

(52) U.S. Cl. 424/468; 424/470; 514/217

(57) ABSTRACT

The present invention relates to multiple-unit modified release carbamazepine compositions for oral administration which include: (i) at least one extended release unit, and (ii) at least one enteric release unit. Also provided are processes for the preparation of multiple-unit modified release compositions of carbamazepine.

### TECHNICAL FIELD OF THE INVENTION

The present invention relates to multiple-unit modified release carbamazepine compositions for oral administration which include: (i) at least one extended release unit, and (ii) at least one enteric release unit. Also provided are processes for the preparation of multiple unit modified release compositions of carbamazepine.

### CROSS REFERENCE TO RELATED APPLICATIONS

This application claims priority under 35 U.S.C. 119(a) from Indian Patent Applications 1380/DEL/2005, filed May 30, 2005 and 1382/DEL/2005, filed May 30, 2005. The entirety of both applications is incorporated herein by reference.

### BACKGROUND OF THE INVENTION

Carbamazepine, chemically described as 5H-dibenz-[b,f]azepine-5-carboxamide, is a well established anti-epileptic compound. It is regarded as a first-line drug in the treatment of patients suffering from partial seizures, with and without second generalization, and in patients with generalized tonic-clonic seizures. Besides being an antiepileptic compound, carbamazepine has also proved effective in the treatment of trigeminal neuralgia and in patients suffering from manic-depressive illness, post therapeutic neuralgia, or phantom limb pain. The drug appears to act by reducing postsynaptic responses and by blocking post-tetanic potentiation.

Although the half-life of carbamazepine is relatively long, ranging between 25 and 85 hours after a single dose, its effect is substantially reduced after repeated dosing due to autoinduction. Due to its increased metabolism, pronounced daily fluctuations in the serum concentration of carbamazepine are observed and are a cause for concern. The therapeutic blood serum concentration range of carbamazepine is about 4-12 μg/ml. However, blood levels of carbamazepine below 4 μg/ml have been found to be ineffective in treating clinical disorders and conversely blood levels greater than 12 μg/ml have been found to increase the chances of side-effects, such as neuromuscular disorders, cardiovascular and gastrointestinal effects. Currently the dosage regimes for conventional carbamazepine formulations typically require 3-4 doses per day to maintain effective blood concentration. This is very bothersome for ambulatory patients, and often times leads to poor patient compliance.

Sustained release dosage forms have been the focus of research for improved therapy, both through improved patient compliance and decreased incidences of adverse drug reactions. It is the intent of sustained release formulations to provide a longer period of pharmacological action after administration than is ordinarily obtained after administration of immediate-release dosage forms. Sustained release compositions may also be used to delay absorption of a medicament until it has reached certain areas of the alimentary tract, and maintain a desired concentration of the medicament in the blood stream for a longer duration than would occur if conventional rapid release dosage forms are administered. Such longer periods of response provide for many therapeutic benefits may not be achieved with corresponding short acting, immediate release preparations. A further general advantage of longer acting drug preparations is improved patient compliance resulting from the avoidance of missed doses through patient forgetfulness.

Sustained release formulations known in the art include specially coated pellets, coated tablets and capsules, and ion exchange resins, wherein the slow release of the active medicament is brought about through selective breakdown of the coating of the preparation or through formulating with a polymeric matrix to affect the release of a drug. Some sustained release formulations provide for pulsatile and/or sequential release of a single dose of an active compound at predetermined periods after administration.

For sustained-release dosage forms containing very high quantities of the active pharmaceutical ingredient, it is particularly critical to avoid an excessively rapid release (dose dumping) as that can lead to undesirable toxic effects. Moreover, such systems are dependent upon gastric emptying rates and transit times, and can be associated with significant intra-and inter-individual variations.

These disadvantages have led to a shift in modified release technology from the use of monolithic systems to multiple unit systems in which each individual unit is formulated with modified release characteristics. The final dosage form includes a multiplicity of the individual units contained in a formulation in such a form that these individual units are made available from the formulation upon reaching the tract.

Multiple unit dosage forms possess a large surface area, which advantageously promotes complete and uniform absorption, minimizes peak plasma fluctuations and thus reduces the potential for systemic side effects. A further advantage of these dosage forms is that high local concentrations of the active substance in the system are avoided as a consequence of the units being distributed freely throughout the tract. The multiple unit dosage form ensures incorporation of higher dose resulting in a decreased dosing frequency and consequently better patient compliance.

JP 61/044811 describes a granulate mixture, which is composed of an initial release portion and a delayed release portion. In the initial release portion the active ingredient is immediately released in the stomach. In the delayed release portion, the granulate is enclosed in a membrane which is resistant to gastric juices. In an alternative embodiment, the active ingredient is blended homogenously with the material resistant to gastric juices.

EP-A-255002 discloses a combination of an initial release granulate mixture with delayed release active ingredient.

Currently, there are a limited number of slow release oral carbamazepine dosage forms available (TEGRETOL®-XR of Novartis and CARBATROL® of Shire Laboratories, Inc.). TEGRETOL®-XR are extended-release tablets available in the U.S. and contain a core and a shell. The contents of the core are released through a small opening on one side. Fluid is absorbed through the shell, causing the contents to expand and slowly push out through the opening. In the United Kingdom, TEGRETOL Retard tablets are available, which contain coated pellets to deliver the medicine. CARBATROL® (extended-release) is a multi-component capsule formulation containing three different types of beads: immediate release, extended-release and enteric-release beads. The three different beads are combined in a specific ratio to provide twice-daily dosing.

U.S. Pat. No. 4,857,336 and RE 34,990, disclose a therapeutic system for peroral administration of carbamazepine. The system comprises a wall made of a material permeable to water and impermeable to the components of the drug-containing core; a core containing finely particulate carbamazepine, a protective colloid, a swellable hydrophilic polymer and an optional water-soluble compound; and a passageway through the wall for delivering the core components to the environmental body fluid. The passageway is produced by mechanical or laser drilling of the outer wall.

U.S. Pat. No. 5,326,570 discloses an extended-release drug delivery system for the oral administration of carbamazepine and a method of treating a patient with the drug delivery systems. The drug delivery systems include a single dosage form containing three types of units: an immediate release unit, a sustained release unit and an enteric release unit capable of releasing carbamazepine at varying times.

U.S. Pat. No. 5,912,013 discloses a composition for treating a patient with carbamazepine in a pharmaceutical dosage form which includes pellets containing at least 70% carbamazepine and 5% of polyvinyl pyrrolidone. Three different types of pellets are prepared, one of which is an immediate release, the second is a slow release and the third is a pH dependent formulation. The three different types of pellets are combined into a single dosage form.

U.S. Pat. No. 5,980,942 describes a zero order sustained release matrix tablet formulation of carbamazepine. The matrix tablet formulation comprises a hydrophilic polymer gel that inhibits transformation of carbamazepine into carbamazepine dihydrate and effectively changes the anhydrous carbamazepine into an amorphous form that can be released from the matrix by zero-order release kinetics.

U.S. Pat. No. 6,294,201 discloses an osmotic drug release system consisting of a shell and core containing a pharmaceutically active substance, xanthan and a vinyl pyrrolidone-vinyl acetate copolymer. These water-expandable polymers allow for the release of the active substance from the shell in a controlled manner.

U.S. Pat. No. 6,475,493 and U.S. Pat. No. 6,635,680 disclose a coating composition comprising a heterogeneous coating mixture of three different polymers; water insoluble polymer, enteric polymer and water soluble polymer. The pharmaceutical formulation coated with this heterogeneous coating mixture provides initiation of the release of the active in the stomach at a slow rate and controls the release in the intestines at a rate faster than that in the stomach such that the active is delivered over the course of predetermined interval.

These methods of carbamazepine delivery, in addition to being expensive, involve time-consuming methods of production. There is a need in the art to develop drug formulations which provide a therapeutically effective blood concentration level of carbamazepine for a sustained period and that involve simple methods of production.

### SUMMARY OF THE INVENTION

In one general aspect there is provided a multiple-unit modified release carbamazepine composition for oral administration. The composition includes:
(i) at least one extended release unit comprising carbamazepine, one or more rate-controlling polymers and one or more pharmaceutically acceptable excipients, and
(ii) at least one enteric release unit comprising a coating of one or more enteric polymers over an extended release or immediate release core of carbamazepine.

Embodiments of the present composition may include one or more of the following features. For example, the ratio of extended release units to the enteric release units may include a range from about 20:80 to about 80:20 by weight.

The extended release core may include carbamazepine, one or more rate-controlling polymers and one or more pharmaceutically acceptable excipients. The immediate release core may include carbamazepine and one or more pharmaceutically acceptable excipients.

The one or more rate-controlling polymers may be cellulose derivatives, starch, polyvinyl pyrrolidone, gums, alginates and acrylic acid derivatives and the enteric polymers may be one or more of cellulose acetate phthalate, cellulose acetate trimellitate, hydroxypropyl methylcellulose acetate phthalate, polyvinyl acetate phthalate, hydroxypropyl methylcellulose phthalate, hydroxypropyl methylcellulose acetate succinate; methacrylic acid copolymers such as Eudragit L 100-55, D-55, 100, and Eudragit S 100, and mixtures thereof.

The one or more pharmaceutically acceptable excipients may be diluents, binders, lubricants, glidants, surfactants, pH-modifiers and colorants.

The multiple-units are formulated as one or more of spheroids, beads, microspheres, seeds, granules, pellets and ion-exchange resin beads. The multiple-units may be filled into capsules or sachets or compressed into tablets.

The coating layer may include one or more of plasticizers, coloring agents, lubricants and anti-adherents.

The modified release carbamazepine composition may be designed to release carbamazepine for up to about 12 hours.

In another general aspect there is provided a process for preparing multiple-unit modified release composition of carbamazepine. The process includes the steps of:
(i) preparing the extended release unit comprising carbamazepine;
(ii) preparing the enteric release unit by providing an enteric polymer coating over a core comprising carbamazepine; and
(iii) mixing the extended release and enteric release units in a ratio of about 20:80 to about 80:20 by weight and filling the units into a capsule or compressing into a tablet.

Embodiments of the process may include one or more of the following features. For example, the extended release unit may be prepared by spraying the blend which includes carbamazepine and one or more rate-controlling polymers onto inert cores.

The extended release unit may be prepared by blending carbamazepine with one or more rate-controlling polymers and one or more pharmaceutically acceptable excipients, wherein blending is carried out by simple granulation followed by sieving; extrusion and marumerization or spheronization, rotogranulation, pelletization and micropelletization.

The core of the enteric release unit may be an extended release core or an immediate release core. The extended release core may be prepared by blending carbamazepine with one or more rate-controlling polymers and one or more pharmaceutically acceptable excipients or by spraying the blend comprising carbamazepine and one or more rate-controlling polymers onto inert cores. The immediate release core may be prepared by blending carbamazepine with one or more pharmaceutically acceptable excipients or by layering carbamazepine over inert cores.

In yet another general aspect there is provided a method of treating convulsions or trigeminal neuralgia. The method includes administering a multiple unit modified release carbamazepine composition which includes:
(i) at least one extended release unit, and
(ii) at least one enteric release unit.

Embodiments of the method may include one or more of following features or those described above. For example, the multiple unit modified release carbamazepine composition further may include one or more additional anticonvulsant or pharmaceutical agents.

### DETAILED DESCRIPTION OF THE INVENTION

The inventors have now developed a modified dosage form of carbamazepine that helps to achieve the desired release profile up to about 12 hours time period. The inventors have also found that the use of a combination of extended release units and enteric release units provide a better control over the rate of release of drug.

The term “modified release” as used herein includes any type of modified release profile including prolonged release, sustained release, controlled release and extended release.

The term “unit” as used herein includes spheroids, beads, microspheres, seeds, granules, pellets, ion-exchange resin beads and other multi-particulate systems.

The modified release carbamazepine composition in unit dosage form of the present invention includes:
(i) at least one extended release unit which includes carbamazepine, one or more rate-controlling polymers and one or more pharmaceutically acceptable excipients, and
(ii) at least one enteric release unit which includes a coating of an enteric polymer over an extended release or immediate release core of carbamazepine, wherein
   the extended release core includes carbamazepine, one or more rate-controlling polymers and one or more pharmaceutically acceptable excipients, and the immediate release core includes carbamazepine and one or more pharmaceutically acceptable excipients.

The ratio of extended release units to enteric release units in the modified-release composition may range from about 20:80 to about 80:20 by weight. The multiple-units are filled into capsules, sachets or compressed into tablets.

The extended release units containing carbamazepine may be formulated by blending carbamazepine with one or more rate-controlling polymers and one or more pharmaceutically acceptable excipients, by the processes known in the art, such as, simple granulation followed by sieving; extrusion and marumerization or spheronization; rotogranulation; pelletization; micropelletization, etc. Alternatively, the blend which includes carbamazepine and one or more rate-controlling polymers is layered onto inert cores as a powder, suspension or solution in a suitable solvent.

The enteric release units may be prepared by providing a coating of one or more enteric polymers over a core which includes carbamazepine. The core may be an extended-release unit or immediate release unit that includes carbamazepine.

The immediate release units containing carbamazepine may be formulated as a plurality of discrete or aggregated particles, pellets, beads or granules. The process for preparing the units may be accomplished by blending carbamazepine with or without one or more pharmaceutically acceptable excipients using any processes known in the art, such as, simple granulation followed by sieving; extrusion and marumerization or spheronization; rotogranulation; pelletization; micropelletization, etc. Alternatively, the immediate release units are formulated by layering carbamazepine with or without pharmaceutically acceptable excipients over inert cores. The active is layered over the inert cores as powder; or as a suspension or solution in a suitable solvent.

The inert core may be hydrosoluble or hydroinsoluble. Examples of inert cores include sucrose, lactose, maltodextrin, microcrystalline cellulose, pergelatinized starch, dicalcium phosphate, celphere and non-pareils. The cores may be of any geometric shape, although spheres are particularly used for the case of uniform coating.

The rate controlling polymers may include one or more of pharmaceutically acceptable polymers that can control the rate of release of carbamazepine, e.g., cellulose derivatives, starch, polyvinyl pyrrolidone, gums, alginates and acrylic acid derivatives.

Suitable examples of cellulosic polymers include ethylcellulose, hydroxypropyl methylcellulose, hydroxypropyl cellulose, methylcellulose, carboxymethylcellulose, hydroxymethylcellulose and hydroxyethylcellulose.

Suitable examples of acrylic acid derivatives include polymethacrylates, such as ethyl acrylate/methyl methacrylate copolymer (Eudragit NE-30-D) and ammonio methacrylate copolymer types A and B (Eudragit RL30D and RS30D).

The enteric release unit is prepared by providing a coating of enteric polymers over a core comprising carbamazepine. The enteric polymers are selected from any such pharmaceutically acceptable enteric polymers that would facilitate erosion and breakdown of the pellets at a pH of 4.5 and above.

Suitable enteric polymers include one or more of cellulose acetate phthalate, cellulose acetate trimellitate, hydroxypropyl methylcellulose acetate phthalate, polyvinyl acetate phthalate, hydroxypropyl methylcellulose phthalate, hydroxypropyl methylcellulose acetate succinate; methacrylic acid copolymers, such as Eudragit L 100-55, D-55, 100, and Eudragit S 100, and mixtures thereof.

Suitable solvents used for preparing a solution of enteric polymers and solution/suspension of active layer include one or more of alcohols, such as ethyl alcohol or isopropyl alcohol; ketones, such as acetone or ethylmethyl ketone; halogenated hydrocarbons, such dichloro ethane or trichloromethane; or mixture thereof.

The coating may be done using a conventional coating pan, a spray coater, a rotating perforated pan, or an automated system, such as a centrifugal fluidizing (CF) granulator, a fluidized bed process, or any other suitably automated coating equipment.

Immediate release units may additionally include one or more surfactants and pH-modifiers.

Suitable surfactants may be anionic, cationic, zwitterionic and nonionic surfactants. The compositions may include at least one anionic surfactant. Suitable anionic surfactants include one or more of alkyl sulfonates, alkyl phosphates, alkyl phosphonates, potassium laurate, sodium lauryl sulfate, sodium dodecylsulfate, alkyl polyoxyethylene sulfates, dioctyl sodium sulfosuccinate, phosphatidyl glycerol, phosphatidylinositol, diphosphatidylglycerol, phosphatidyl inosine, phosphatidylserine, phosphatidic acid and their salts, cholic acid and other bile acids (e.g., cholic acid, deoxycholic acid, glycocholic acid, taurocholic acid, glycodeoxycholic acid) and salts thereof (e.g., sodium deoxycholate, etc.).

Suitable pH-modifiers may include one or more of citric acid, sodium bicarbonate, monosodium citrate, trisodium citrate, tribasic sodium phosphate, sodium chloride or mixtures thereof.

The units may also include pharmaceutically acceptable excipients, which act in one or more capacities as diluents, binders, lubricants, glidants or colorants.

Suitable diluents may include one or more of corn starch, lactose, white sugar, sucrose, sugar compressible, sugar confectioners, glucose, sorbitol, calcium carbonate, calcium phosphate-dibasic, calcium phosphate-tribasic, calcium sulfate, microcrystalline cellulose, silicified microcrystalline cellulose, cellulose powdered, dextrates, dextrins, dextrose, fructose, kaolin, lactitol, mannitol, starch and starch pregelatinized.

Suitable binders include one or more of methyl cellulose, hydroxypropyl cellulose, hydroxypropyl methylcellulose, polyvinylpyrrolidone (povidone), copolymer of polyvinylpyrrolidone and vinyl acetate (copovidone), gelatin, gum arabic, ethyl cellulose, polyvinyl alcohol, pullulan, pregelatinized starch, agar, tragacanth, sodium alginate, and propylene glycol.

Suitable lubricants and glidants include one or more of colloidal anhydrous silica, stearic acid, magnesium stearate, calcium stearate, talc, hydrogenated castor oil, sucrose esters of fatty acids, microcrystalline wax, yellow beeswax and white beeswax.

A coating layer may additionally include one or more of plasticizers, coloring agents, lubricants, antiadherents, and mixtures thereof.

Suitable plasticizers include one or more of propylene glycol, triethylene glycol, oleic acid, triethylcitrate, tributylcitrate, triacetin, diethyl phthalate, dibutyl phthalate, dibutylsebacate, glyceryl monostearate, castor oil, ethylene glycol monooleate, and mixtures thereof.

The coloring agents and flavoring agents of the present invention may be selected from any FDA approved colors and flavors for oral use.

Also provided is a method of treating convulsions or trigeminal neuralgia, by administering a multiple unit modified release carbamazepine composition. The method includes administering a pharmaceutical composition that includes:
(i) at least one extended release unit, and
(ii) at least one enteric release unit.

The multiple unit modified release carbamazepine composition for oral administration that includes (i) at least one extended release unit, and (ii) at least one enteric unit, may be administered in combination with other medicines, for example, other anti epileptic drugs, like lithium carbonate, phenobarbitone, sodium valporate, phenyloin, gabapentin, lamotrigine, etc.

The multiple units of modified release carbamazepine composition are filled into capsules or sachets or compressed into tablets.

The following non-limiting examples further illustrate the modified release formulations of carbamazepine and processes of making such formulations.

### EXAMPLE 1

### Part A: Preparation of Extended Release Units

| Ingredients | Percent w/w |
|---|---|
| Carbamazepine | 76.5 |
| Microcrystalline Cellulose | 16.5 |
| Hydroxypropyl methylcellulose | 4.7 |
| Copolymer of polyvinylpyrrolidone | 2.3 |
| and vinyl acetate (Copovidone) | |
| Water | q.s. |

1. Carbamazepine, hydroxypropyl methylcellulose and microcrystalline cellulose were granulated with an aqueous solution of copolymer of polyvinylpyrrolidone and vinyl acetate (Copovidone) in water.
2. The wet mass was extruded, spheronized, dried and sieved to get spherical units.

### Part B: Preparation of Enteric Release Units

| Ingredients | Percent w/w |
|---|---|
| Carbamazepine | 71.0 |
| Microcrystalline Cellulose | 19.0 |
| Lactose | 4.7 |
| Citric acid | 2.4 |
| Sodium lauryl sulphate | 2.4 |
| Copolymer of polyvinylpyrrolidone | 0.5 |
| and vinyl acetate (Copovidone) | |
| Water | q.s. |

Coating composition

| | |
|---|---|
| Eudragit L 30 D 55 | 54 |
| Talc | 39 |
| Triethyl Citrate | 5.5 |
| Colloidal Silicon dioxide | 1.5 |
| Water | q.s. |

1. Carbamazepine, microcrystalline cellulose, lactose, citric acid and sodium lauryl sulphate were granulated with aqueous solution of copolymer of polyvinylpyrrolidone and vinyl acetate (Copovidone) in water.
2. The wet mass was extruded, spheronized, dried and sieved to get spherical units.
3. The spherical units of step 2 were coated with the coating composition provided in the table up to a weight build up of about 15%.

### Part C: Preparation of Modified release carbamazepine compositions

The exteneded release units and enteric release units were blended in a desired ratio and filled into capsules.

### Example 2

### Preparation of Modified Release carabamazepine compositions

| Ingredients | Percent w/w |
|---|---|
| Carbamazepine | 76.5 |
| Microcrystalline Cellulose | 17.6 |
| Hydroxypropyl methylcellulose | 5.9 |
| Water | q.s. |

Coating composition

| | |
|---|---|
| Eudragit L 30 D 55 | 54 |
| Talc | 39 |
| Triethyl Citrate | 5.5 |
| Colloidal Silicon dioxide | 1.5 |
| Water | q.s. |

1. Carbamazepine, hydroxypropyl methylcellulose and microcrystalline cellulose were granulated with water.
2. The wet mass was extruded, spheronized, dried and sieved to get spherical extended release units.
3. A part of the prepared extended release units of step (2) were coated with the coating composition provided in the table up to a weight build up of about 15% to prepare enteric release units.
4. The extended release units of step (2) and the enteric release units of step (3) were blended in a desired ratio and filled into capsules.

While several particular forms of the inventions have been described, it will be apparent that various modifications and combinations of the inventions detailed in the text can be made without departing from the spirit and scope of the inventions. Accordingly, it is not intended that the inventions be limited, except as by the appended claims.

We claim:

1. A multiple-unit modified release carbamazepine composition for oral administration comprising:
(i) at least one extended release unit comprising carbamazepine, one or more rate-controlling polymers and one or more pharmaceutically acceptable excipients, and
(ii) at least one enteric release unit comprising a coating of one or more enteric polymers over an extended release or immediate release core of carbamazepine.

2. The composition according to claim 1, wherein the ratio of extended release units to the enteric release units comprises a range from about 20:80 to about 80:20 by weight.

3. The composition according to claim 1, wherein the extended release core comprises carbamazepine, one or more rate-controlling polymers and one or more pharmaceutically acceptable excipients.

4. The composition according to claim 1, wherein the immediate release core comprises carbamazepine and one or more pharmaceutically acceptable excipients.

5. The composition according to claim 1, wherein the one or more rate-controlling polymers comprise cellulose derivatives, starch, polyvinyl pyrrolidone, gums, alginates and acrylic acid derivatives.

6. The composition according to claim 1, wherein the enteric polymers comprise one or more of cellulose acetate phthalate, cellulose acetate trimellitate, hydroxypropyl methylcellulose acetate phthalate, polyvinyl acetate phthalate, hydroxypropyl methylcellulose phthalate, hydroxypropyl methylcellulose acetate succinate; methacrylic acid copolymers such as Eudragit L 100-55, D-55, 100, and Eudragit S 100, and mixtures thereof.

7. The composition according to claim 1, wherein the one or more pharmaceutically acceptable excipients comprise one or more of diluents, binders, lubricants, glidants, surfactants, pH-modifiers and colorants.

8. The composition according to claim 1, wherein the multiple-units are formulated as one or more of spheroids, beads, microspheres, seeds, granules, pellets and ion-exchange resin beads.

9. The composition according to claim 1, wherein the multiple-units are filled into capsules or sachets or compressed into tablets.

10. The composition according to claim 1, wherein the coating layer comprises one or more of plasticizers, coloring agents, lubricants and anti-adherents.

11. The composition according to claim 1, wherein the modified release carbamazepine composition is designed to release carbamazepine for up to about 12 hours.

12. A process for preparing multiple-unit modified release compositions of claim 1 comprising the steps of:
(i) preparing at least one extended release unit comprising carbamazepine;
(ii) preparing at least one enteric release unit by providing an enteric polymer coating over a core comprising carbamazepine; and
(iii) mixing the extended release and enteric release units in a ratio of about 20:80 to about 80:20 by weight and filling the units into a capsule or compressing into a tablet.

13. The process according to claim 12, wherein the extended release unit is prepared by spraying the blend comprising carbamazepine and one or more rate-controlling polymers onto inert cores.

14. The process according to claim 12, wherein the extended release unit is prepared by blending carbamazepine with one or more rate-controlling polymers and one or more pharmaceutically acceptable excipients and the blending is carried out by simple granulation followed by sieving; extrusion and marumerization or spheronization, rotogranulation, pelletization and micropelletization.

15. The process according to claim 12, wherein the core of the enteric release unit is an extended release core or an immediate release core.

16. The process according to claim 15 wherein the extended release core is prepared by blending carbamazepine with one or more rate-controlling polymers and one or more pharmaceutically acceptable excipients or by spraying the blend comprising carbamazepine and one or more rate-controlling polymers onto inert cores.

17. The process according to claim 15, wherein the immediate release core is prepared by blending carbamazepine with one or more pharmaceutically acceptable excipients or by layering carbamazepine over inert cores.

18. A method of treating convulsions or trigeminal neuralgia, the method comprising administering a multiple unit modified release carbamazepine composition of claim 1 comprising at least one extended release unit, and at least one enteric release unit.

19. The method of treatment according to claim 18, wherein the multiple unit modified release carbamazepine composition further comprises one or more additional anticonvulsant or pharmaceutical agents.
